(19) United States
(12) Patent Application Publication
(10) Pub. No.: US 2002/0160060 A1
(43) Pub. Date: Oct. 31, 2002

(54) ENRICHED SPREADS

(76) Inventors: MANDY KIM CHEN, BALTIMORE, MD (US); MATTHEW PATRICK, ANNAPOLIS, MD (US); PODUTOORI RAVINDER REDDY, COLUMBIA, MD (US)

Correspondence Address: UNILEVER, PATENT DEPARTMENT, 45 RIVER ROAD, EDGEWATER, NJ, 07020, US

(21) Appl. No.: 09/299,778

(22) Filed: Apr. 26, 1999

(51) Int. Cl. A61K 35/78; A23K 1/00

(52) U.S. Cl. 424/757; 426/615

(57) ABSTRACT

In accordance with a first aspect of the invention, it has now been found that a beneficial form for ingestion of phytoestrogens is in the form of a water-in-oil spread. For instance, it has been discovered that phytoestrogens can advantageously be consumed, particularly in elevated amounts, when included in the form of a bread spread. It can be expected that the reported beneficial health effects of phytoestrogens may be enjoyed by the consumer by consuming the spread without the need for pharmaceutical type pills, capsules, etc. Moreover, the spreads of the invention have good taste, notwithstanding the presence of the often-bitter tasting isoflavones.

### BACKGROUND OF THE INVENTION

Phytoestrogens are compounds found in plants which have certain estrogen-like effects. These include isoflavones, lignans, flavones and coumestans. They have been touted in the press for beneficial health effects without certain disadvantages which have been attributed to estrogen, such as increased risk of cancer. Health conditions for which phytoestrogens have been mentioned include cancer of the breast, cancer of the prostate, cancer of the uterus, cancer of the bowel, hypercholesterolemia, atherosclerosis, colon cancer, antioxidation, benign breast disease, premenstrual syndrome, and symptoms associated with menopause.

Phytoestrogens are weakly estrogenic and competitively inhibit the response of tissue to estrogens. They have been found in vitro to stimulate production of sex hormones-binding globulin (SHBG) from human cells. Phytoestrogens are described in Price et al. “Naturally occuring oestrogens in foods—A review,” Food Additives and Contaminants,” 1985, Vol. 2, NO. 2, 73-106, the disclosure of which is incorporated by reference herein.

Numerous references exist in the patent and scientific literature to phytoestrogens and related compounds.

Crank et al. U.S. Pat. No. 5,858,449 is directed to isoflavone-enriched soy protein products and methods for their manufacture. One of the key factors said to limit the use of soy protein isolates especially in nutritional beverages, milk replacers, and dairy products is the soy taste. Soy concentrates are said not to be used in such products because of the strong soy taste and the presence of the soy fiber as an insoluble component with undesirable mouthfeel.

Crank's finished product is said to display desirable flavor and functional properties. The isoflavone content is said to be substantially increased compared to traditional soy protein isolates. The product may be an ingredient in dairy or meat-based food products such as infant formula, nutritional beverage, milk replacer, bologna, imitation processed cheese spread, water-injected ham, yogurt and frozen dessert. The Crank et al. soy product has a protein content greater than 60% dry matter, dietary fiber less than 4% dry matter, sucrose greater than 10% dry matter, sulfur containing amino acid greater than 2.2% of total amino acid content, stachyose content less than 1.5% dry matter, and total isoflavone content greater than 2500 mcg/g.

Crank et al. also disclose a method of making an isoflavone enriched soy product.

It is said that the soy should be present as an ingredient in cheese spread at from 10-50% of the protein used in the formula. Example 5 discloses a soy based imitation processed cheese spread. Ingredients include vegetable oil, casein, sodium citrate, disodium phosphate, sodium aluminum phosphate and whey. Example 8 discloses a soy-based frozen dessert and a yogurt. The dessert includes hydrogenated soybean oil, cellulose gum, CMC, locust bean gum, xanthan gum and carrageenan. The yogurt includes whey, vegetable oil, sugar, emulsifiers, salts, vitamins and minerals. The soybean products include daidzein, genistein, and glycitein.

Kelly, U.S. Pat. No. 5,830,807 is directed to compositions enriched with natural phytoestrogens or analogs thereof selected from genistein daizein, formonetion and biochanin A. It is said that they may be used as food additives, tablets or capsules for promoting health in cases of cancer, premenstrual syndrome, menopause or hypercholesterolaemia. Soy flour could be used or raw hypocotyl, and whole material can be ground or milled to produce a dry powder or flour which could be added to other prepared foodstuffs. Kelly discloses that isoflavones are intensely astringent. Formulations may include drinks, solutions, syrups, etc.

WO 98/08503 discloses administration of an isoflavone-type compound used for various conditions including menopausal syndrome such as hot flashes, anxiety and depression, moods swings, night sweats, headaches, urinary incontinence, osteoporosis, premenstrual syndrome, fluid retention, cyclic mastalgia dysmenorrhea, Raynaud's syndrome, Raynaud's phenomenon and Buerger's diseases, coronary artery spasms, migraine headaches, hypertension, benign prostatic hypertrophy, cancers of the breast, uterus, ovary, colon, endometrium, testicle, prostate, or large bowel, cyclical mastalgia, aetheroscierosis, Alzheimer's disease, male impotency, inflammatory bowel syndrome, ulcerative colitis, Crohn's disease, inflammatory effects such as rheumatoid diseases including rheumatoid arthritis, acne, baldness, including male pattern baldness, psoriasis and diseases associated with oxidant stress including cancer, myocardial infarction, stroke, arthritis, sunlight induced skin damage or cataracts, and oxidant related disorders, inflammatory diseases, menopausal syndrome, anxiety, depression, mood swings, acne, estrogenic effects, androgenic effects, vasodilatory and spasmodic effects. It can be used with vitamin E. Use as additives in foods and drinks such as health bars or desserts is mentioned.

Among the many forms of formulation suitable for oral administration mentioned are oil-in-water and water-in-oil emulsions. It is said that the formulations may be prepared by any suitable method of pharmacy which involves the step of bringing into association the active compound and a suitable carrier. The term “food stuffs” is said to be used in as wide as possible sense. It includes liquid formulations such as drinks, including dairy products, and other foods. Food formulations, it is said, can be readily prepared according to standard practices. Health drinks are mentioned. The compounds are said to act synergistically with vitamin E to protect biological molecules from oxidation. In Example 5, therapeutic formulations were prepared by admixing compounds with a soy flour base.

Gorbach et al, U.S. Pat. No. 5,498,631 is directed to a method for treating symptoms of menopause, premenstrual syndrome or a condition resulting from reduced levels of indigenous estrogen by administering to the women an effective amount of an isoflavoid. Isoflavonoids which may be administered include genistein, daizein, biochanin A, formonetin, o-desmethylangolensin and equol. These may be administered alone or in combination. The invention is said to feature a therapeutic dietary product for preventing or treating symptoms resulting from reduced or altered levels of indigenous estrogen. The dietary products preferably include a soy extract containing enriched isoflavonoids provided in a palatable food carrier, (e.g. a confectionery bar, biscuit, cereal or beverage).

Gorbach U.S. Pat. No. 5,733,926 is directed to a method for treating Alzheimer's disease or age-related loss of cognitive function comprising administering an isolated isoflavonoid selected from genistein, daidzein, biochanin A, formononetin, O-desmethylangolensin, glycitin and equol. It may be provided in a food carrier such as a confectionary bar, biscuit, cereal or beverage.

Sekiya et al., U.S. Pat. No. 5,776,906 is directed to a method for promoting fat degradation comprising administrating to a human a composition containing an effective amount of an isoflavone, thereby promoting fat degradation in the fat cell. Soybean is mentioned as a potential source and daidzein, daidzin, genistein, genistin and derivatives thereof are mentioned. When the composition is intended to be a food it contains five to 1,000 milligrams-milliliter (g) of isoflavone. Any commercially available isoflavones may be obtained, but those obtained from a soybean using extraction may be employed. The extraction may be conducted using an organic solvent such as methanol. Composition may be a pharmaceutical cosmetic functional food or the like.

Barnes et al., U.S. Pat. No. 5,506,211 discloses that the isoflavone genistein inhibits the acid secretion of osteoclasts and reduces bone resorption. The claims mention use of a genistein/glucoside conjugate. To reduce osteoclastic acid secretion one would generally contact one or more osteoclasts with a composition that comprises a biologically effective amount of genistein. Foodstuffs such as soy which contain genistein or concentrated forms thereof may be ingested to provide an animal with an effective amount of genistein. Conjugated forms of genistein are absorbed in the large bowel and thus are said to have advantageous biodistribution properties. Various soy products such as soy protein may be used.

The Barnes invention is said to relate to novel methods for reducing bone resorption to subjects that exhibit symptoms associated with bone resorption such as those known or suspected to have bone cancer or a cancer that has metastasized to the bone, patients with various hypercalcemias, patients with Paget disease and patients with advanced end stage renal disease. The treatment methods of the invention comprise administering to the subject a pharmaceutically acceptable genistein composition in an amount that is effective to reduce acid secretion by osteociast located within the animal. In human treatments, suitable methods include administering from two to 50 milligrams to 20 to 50 milligrams of genistein in the form of a food product. This may be achieved by ingesting between by about two to 50 milligrams or about 20 to 50 milligrams of soy isolated soy protein per day per person. Barnes acknowledge that genistein is known to be a tyrosine kinase inhibitor and has been proposed for use in treating several diseases and disorders, for example cardiovascular disease, athleroscerlosis and certain cancers.

Barnes et al. admit that isoflavones in most of the common soy foods such as soy milk, tofu, soy flour, soy protein concentrate and soy protein isolate are present as glycosidic conjugates. The Barnes et al. inventors are said to have found that in the unprocessed soybean hypocotyl and cotyledon, the isoflavones are found in beta glucosides, 6-0″-malonylgucosides and 6-0″-acetylglucosides. Soy flour prepared by hexane extraction of crushed soybeans are said to contain mostly 60 MalGlc conjugates. Increasing use of heat is said to lead to increased though small amounts of 60 AcGlc conjugates.

Barnes et al. indicate that soy foods are not necessarily of equivalent nutritional or therapeutic value because the different conjugate forms have been discovered to have quite different roots of uptake leading to different bioavailabilities. Barnes et al. indicate that genistein when conjugated to an organic acid is not readily hydrolyzed in the stomach and is not available for significant absorption in the small intestine. Thus such a form of genistein is mainly absorbed through the large bowel. As an example is given the consumption of a product high in 6″-0-malonoglucoside conjugates of isoflavones, the isoflavones not being detected in the urine or serum from 4-8 hours later, indicating that the principal site of intestinal absorption is the large bowel. On the other hand, consumption of a full fat soy milk containing beta glucoside conjugates only leads to the detection of isoflavones in the serum within two hours suggesting that absorption occurs in this case from the small intestine. Therefore, Barnes et al. propose that using soy compositions having higher quantities of conjugated genistein, i.e. non heat treated soy products or isolated soy protein, allows genistein availability to be controlled and genistein to be targeted to the large bowel. This is envisioned to be of importance to treating bowel cancel wherein the use of a soy composition high in conjugated genistein would be a significant advantage.

Feuer et al., U.S. Pat. No. 4,163,746 discloses 5-methylalkoxy isoflavones useful as weight gain promoters. In the form of pharmaceutical compositions, the compositions may be in solid or liquid form. The liquid may be, for example, an emulsion. The composition contains conventional diluents or carriers such as talc, magnesium stearate, calcium carbonate, starch and water. Optionally other agents such as emulsifying agents may be used. Further biologically active components such as vitamins may be used. In the example at the top of column 5, 26% calcium phosphate, 1% of a vitamin premix and 0.05% of a mineral premix are used. Further additives mentioned include vitamins, amino acids, choline chlorides, salts of mineral acids, trace elements and other known subjects of biological importance.

Tsuda et al., U.S. Pat. No. 4,644,012 (Takeda Chemicals) is directed to certain substituted isoflavones for prevention of osteoporosis.

Stadler Nee Szoke et al., U.S. Pat. No. 4,826,963 is directed to inclusion complexes of 7-isopropoxyisoflavon formed with cyclodextrin. They mention that 7-isopropoxyisoflavon is used for the treatment of osteoporosis and osteomalacia. The inclusion complex according to the Szoke invention enjoys an in vitro solubility ten-fold that of prior preparation of 7-isopropoxyisoflavon. A commercial product iprisoflavon includes 7-isopropoxyisoflavon, talcum, and magnesium.

Jackson et al., U.S. Pat. No. 5,654,011 (Energetics) filed Jul. 30, 1996 is directed to dietary supplement for supplementing the nutritional needs of pre-perimenopausal women comprising of about 200 to about 500 milligrams calcium, about 100 to about 200 milligrams magnesium, about 0.5 to about 1.5 milligrams boron, from about 0.5 to about 1.5 milligrams copper, about 2 to about 2.6 milligrams manganese, about 10 to 13 milligrams zinc, about 200 to about 300 IU Vitamin D, about 12 to 18 milligrams iron, about 400 to about 440 mug folic acid, about 2 to about 10 mug Vitamin B₁₂, about 50 to about 100 milligrams Vitamin B₆, about 50 to about 100 mug chromium, about 100 to about 200 IU Vitamin E, about 100 to about 1,000 mg Vitamin C and about 8 to less than 50 mg. phytoestrogen, in a mixture with a biologically acceptable carrier. The invention is directed to dietary supplements formulated to supplement a woman's specific micronutrient and phytochemical needs. Dietary supplements of the invention may be formulated as a tablet, capsule, powder, gel or liquid or dietary bar. Jackson et al. U. S. Pat. No. 5,807,586 also discloses vitamins, plus minerals plus phytoestrogens.

Levy et al., U.S. Pat. No. 5,780,060 (Centre National Bela Recherche Scintifigue) is directed to microcapsules based on crosslinked plant polyphenols, particularly flavonoids. These may be incorporated in a composition such as a cosmetic, pharmaceutical, dietetic or food composition. The anti-free radical and/or anti-oxidizing agent of the plant polyphenols is cited. The microcapsules may be formed by interfacial crosslinking of an emulsion dispersed phase contains one or more water soluble liposoluble or insoluble active substances. Active substances can include iron oxide, titanium oxide, zinc oxide, talc or kaolin, a vegetable oil such as cereal germ oil, a deodorized fish liver oil or an oily solution of a lipo soluble substance such as vitamin A, vitamin D2, vitamin E or tocopherol, an essential fatty acid such as linoleic acid, linolenic acid or aichidonaic acid.

Kuznicki et al., U.S. Pat. No. 5,464,619 is directed to composition preferably in the form of a beverage which includes green tea solids, flavanols, sodium ions, potassium ions, and carbohydrate. Minor ingredients which may include flavoring agents, colorings, preservatives, acidulants, gums, emulsifiers, oils and vitamins may be included. Flavoring agents may include various fruit juices. Preferably the vitamins include vitamins A, C and E, although D and B may be included. Additional minerals may be included, such as calcium, chromium, copper, fluourine, iodine, iron, magnesium, maganese, phosphorus, selenium, silicone, molybdenum and zinc.

Kuznicki et al., U.S. Pat. No. 5,464,619 is directed to a composition including flavanols which may be a beverage including green tea solids.

Kelly, U.S. Pat. No. 5,830,887 is directed to compositions enriched with natural phytoestrogens selected from genistein, daizein, formononetin and biochanin A. These may be used as food additives, tablets or capsules for promoting health in cases of cancer, premenstrual syndrome, menopause, or hypercholesterolaemia. In Example 4, soy hypocotyl was consumed as a powder added to the diet.

Teschero, U.S. Pat. No. 5,589,182 is directed to pharmaceutical compositions for cardiovascular disease, cerebral vascular disease, Alzheimers disease, depression or a combinations thereof comprising various mixtures of aqueous extracts of tissue of specific Chinese plants and herbs.

WO 98/50026 (Novogen) discloses treatment or prevention of menopausal symptoms or osteoporosis by administration of isoflavones, formononetin or daizein with one or more adjuvants, carriers and/or excipients.

Liu et al., “A comparison of pharmacodynamics between daizein and solid dispersion of daizein”, Shenyang Yaoxueyuan Xuebao, 1990, Vol. 7, No. 2, 123-5, pp. 131 discloses that daizein is used in clinical treatment of hypertension and coronary atherosclerotic heart disease, but is absorbed so slowly that it begins to show the effects only in a weeks time. A solid dispersion of daizein is disclosed and results are said to show that at equal dosage levels the solids dispersion produced more significant results on arrhythmia induced by BaCl₂ in anesthetized rats.

Zilliken, U.S. Pat. No. 4,157,984 is directed to antioxidant compositions useful as stabilizers for food compositions including edible fats and oils. The compositions are prepared from a natural source, tempeh, a fermented soybean product. An ergostadientriol which possesses antioxidative properties and which in combination with mixtures of isoflavones provides compositions having exceptional antioxidative properties is disclosed. This can be used alone or in mixtures with isoflavones or other compounds.

Shlyankevich, U.S. Pat. No. 5,424,331 (Biovirus Research) is directed to a composition for treatment or prevention of osteoporosis which includes one or more phytoestrogen compounds, calcium contained in a biologically acceptable calcium salt, magnesium contained in a biologically acceptable magnesium salt, zinc contained in a biologically acceptable zinc salt, beta carotene, vitamin D and vitamin E. The compositions may be administered either as a dietary supplement or as a pharmaceutical.

Potter et al., U.S. Pat. No. 5,855,892 is directed to a method of altering the concentration of cholesterol constituents in human blood. The method includes administering a daidzein material as a pharmaceutical composition or in a dietary supplement including soy protein based dietary supplements. Inert pharmaceutically acceptable carriers useful for the invention are said to include calcium sulfate, dicalcium phosphate, magnesium stearate, and disintegrating agents include calcium carbonate. The foods to which daidzein may be added are said to include all foods. Specifically mentioned are beverages such as nutritional beverages, cheeses such as hard and soft cheeses, frozen desserts such as ice cream, salad dressings, and dips and spreads such as mayonnaise and chip dips. In Example 1 the isolated soy proteins containing isoflavones are fortified with calcium.

Shlyankevich, U.S. Pat. No. 5,569,459 is directed to a composition for controlling the stimulation of estrogen production which includes phytoestrogen compounds, a sedative which is a valerian root dry extract, passion flower dry extract, or ginseng root powder, optionally dried licorice root, optionally beta carotene, optionally pyridoxidine hydrochloride, optionally vitamin E, optionally calcium salt, optionally magnesium, optionally zinc, optionally coumestan and optionally pantothenic acid.

Price et al. “Naturally occuring oestrogens in foods—A review,” Food Additives and Contaminants,” 1985, Vol. 2, NO. 2, 73-106. contains a discussion of estrogens in foods.

WO 9610341 (Schouten Industries) discloses substantially pure hypocotyls of Glycine max which may be used in food and other products. They may be used as raw materials for isolation of isoflavones such as daidzin, genistin and glycitin. They may be incorporated in drinks, dairy products, bakery products, health teas and other products. In Example 2, a tomato juice cocktail is disclosed including tomato concentrate, green tea natural beta carotene, natural vitamin E and Glycine max hypocotyl. The product contained 10 mg genisteine/daidzeine as glucosides per can of 163 ml.

WO 9821947 (Internutria) discloses a composition for alleviating persistent reproductive transition nighttime symptoms comprising phytoestrogen and melatonin. The composition can be sprinkled on foods or blended with a food or drink. The food or drink may be solid, semi-solid or liquid. Examples are cereal products, baked goods, puddings, food bars, frozen products such as ice cream, cake mixes or spreads. Some other specific carriers mentioned include sorbet, sherbet, tea, and milk. A dry powder for beverages is found in 6.5.A.

Zilliken U.S. Pat. No. 4,390,559 is directed to isoflavones useful as antioxidants and useful in antioxidant compositions including edible fats and oils. The compounds can be recovered from tempeh, fermented soybean product. Effective amounts of the antioxidant compositions for improving the stability of oils or fats such as lard, corn oil, olive oil, bean oil, safflower oil, vegetable oil, cottonseed oil, polyunsaturated oils, animal fats and oils and the like are said to be in amounts of about 0.01 to 1.0 weight percent, more or less.

Schouten Industries, USA sells a product soybean isolate product called SoyLife® comprising 40.5% protein, 11.2% fatty acid, 3.0% isoflavones, and 4.1% saponins. They suggest incorporating 1% to 5% of the SoyLife® product in any foods, including dietary drinks.

One difficulty which has been encountered in promoting ingestion of phytoestrogens, is the off-taste from which phytoestrogen-containing preparations often suffer.

### SUMMARY OF THE INVENTION

In accordance with a first aspect of the invention, it has now been found that a beneficial form for ingestion of phytoestrogens is in the form of a water-in-oil spread. For instance, it has been discovered that phytoestrogens can advantageously be consumed, particularly in elevated amounts, when included in the form of a bread spread. It can be expected that the reported beneficial health effects of phytoestrogens may be enjoyed by the consumer by consuming the spread without the need for pharmaceutical type pills, capsules, etc. Moreover, the spreads of the invention have good taste, notwithstanding the presence of the often-bitter tasting isoflavones.

In another preferred embodiment, the spread is an emulsion comprising added phytoestrogens and at least 0.25 wt. % of calcium (e.g., calcium moiety of a calcium salt), especially at least 0.5 wt. % of calcium. The spread is preferably a water-in-oil emulsion. The spread is an excellent vehicle to provide women with the phytoestrogens and calcium both of which have enjoyed favorable reports concerning health effects.

In another preferred embodiment of the invention, the spread is an emulsion comprising phytoestrogens and one or more, preferably at least two, of the following vitamins: D, E, B6 and B12. Preferably the spread also includes elevated levels of calcium and/or magnesium. Preferably this spread is also a water-in-oil emulsion.

In another preferred feature of the invention, a spread is provided with phytoestrogens and a level and type of triglycerides such that at least 5 wt. % polyunsaturated fatty acid moieties are present (based on the total weight of the spread) to provide consumers with access to both of these substances in a beneficial food form. Health benefits for both phytoestrogens and polyunsaturated fatty acids have been widely reported. More preferably, the level and type of triglycerides is selected so that the spreads include at least 7 wt. %, especially up to a level of 20 wt. % polyunsaturated fatty acid moieties.

The spreads of the invention also may provide soy proteins in advantageous levels so as not to unduly impair the quality of the product, preferably between 0.4 wt. % and 2 or 3 wt. %.

Isoflavones which may be used include genistein and daidzein.

Advantageously, the spreads of the invention are readily manufactured.

For a more complete understanding of the above and other features and advantages of the invention, reference should be made to the following detailed description of preferred embodiments and to the accompanying drawings.

### DETAILED DESCRIPTION OF THE INVENTION

Preferred sources of phytoestrogen isoflavones include soy, clover, including red clover and subterranean clover, grams, chickpeas, ground nuts, lentils and beans, at levels of between 40 and 300 mg/100 g dry weight. Isoflavones are found in plants primarily bound to sugars such as glucose, as glycosides. Smaller amounts are found in plants in the aglucone form. Soy may be used in the form of soybean flour; or the hull and/or hypocotyl may be used.

Preferred sources of Lignan phytoestrogens are found in common human foods such as oilseeds, eg, flax, cereals, legumes and vegetables. An example is metairesinol. Lignan levels in oilseeds may range from 20-60 mg/100 g dry weight, while other plant sources may range from, e.g., 0.1-0.6 mg/100 g. Coumestans are found in, eg., sprouts of soy and alfalfa at levels ranging between 0.12 mg and 7 mg per 100 g dry weight. Phytoestrogen estrogens are closely related to animal estrogens and have been found in plants such as apple, licorice, and date palm.

Processes for isolating phytoestrogens and phytoestrogen moiety-containing compounds and complexes from plants are well known. These include the process of Fluery et al. U.S. Pat. No. 5,141,746, the disclosure of which is incorporated by reference, Gugger et al. U.S. Pat. No. 5,702,752 and Shen et al. U.S. Pat. No. 5,637,562. Phytoestrogens are also available in the form of soy isoflavone concentrate obtained from soy flour and sold under the Soylife® trade name by Schouten USA, Inc. of Minneapolis, Minn.

Preferred levels of the phytoestrogens are at least 0.01 wt. % on the total weight of the spread, preferably at least 0.05 wt. %.

Other ingredients reported to have healthy benefits which may be included in the spreads are the sterols and sterol esters or stanols and stanol esters such as sitosterol, sitostanol, their fatty acid esters, and the like at up to about 20%, especially up to about 10% of the spread based on the sterol moiety.

Examples include alpha sitosterol, beta sitosterol, stigmasterol, ergosterol and campesterol, alpha spinosterol and brassiciasterol. Although the foregoing are some of the more important phytosterois, at least 44 phytosterols have been identified and it will be apparent to one of ordinary skill that many of these will be appropriate for the present invention. Phytosterols are identified in bean (1993) phytosterols in “Advance in Lipid Research”, pages 193-218, Paoletti, and Kiritchevsky, (Eds) Academic Press, NY, the disclosure of which is incorporated herein by reference. The disclosure of “Effect of Plant Sterols on Lipids and Atherosclerosis”, Pollack, O. J., Pharmac. Ther., 31, 177-208 (1985) mentioned above is also incorporated by reference herein.

Among the more important sources are rice bran, corn bran, corn germ, wheat germ oil, corn oil, safflower oil, oat oil, olive oil, cotton seed oil, soybean oil, peanut oil, black tea, orange juice, valencia, green tea, Colocsia, kale, broccoli, sesame seeds, shea oils, grapeseed oil, rapeseed oil, linseed oil, canola oil, tall oil from wood pulp and other resinous oil from wood pulp.

The spread is advantageously prepared by combining a fat phase with an aqueous phase, after which the mixture is processed into an emulsion and the phytoestrogens and other additives are added.

Spreads according to the invention generally contain from less than 80% be weight of edible triglyceride materials. Suitable edible triglyceride materials are for example disclosed in Bailey's Industrial Oil and Fat Products (1979). In higher fat spreads, the level of triglyceride material will generally be more than 60% and less than 80%, preferably from 70 to 79% by weight. In spreads of reduced fat content the level of triglycerides will generally be from 30-60%, more generally from 35 to 45% by weight. In very low fat spreads the level of triglycerides will generally be from 0 to 40%, for example, 30%, 25%, 20% or even 10% or about 0%.

Optional ingredients in the fat-continuous phase which is combined with the aqueous composition include emulsifiers, salt (particularly sodium chloride), preservatives, flavors, protein, vitamins, especially fat soluble vitamins such as vitamin A., antioxidants, antimicrobials, and preservatives, including citric and other acids. The emulsifiers can include mono-and diglycerides, patial polyglycerol esters, lecithin and polyoxyethylene sorbitan monoesters such as TWEEN 60 and TWEEN 80. One advantageous emulsifier is a polyglycerol polyricinoleate sold under the name Admul Wol available from Quest International, Naarden, the Netherlands.

Emulsifiers may be included at from 0.05 to 2% by weight, typically not more than 1% by weight.

It is preferred that the fat used is triglyceride fat derived from vegetable sources including soybean, canola, corn, sunflower, palm, Palm kernal, rapeseed, coconut, safflower, cottonseed, peanut and olive oils. Other digestible fat sources which may be used are fish oil, milk fat, skim milk fat, butterfat, lard and tallow. The oil will be hardened by hydrogenation if that is necessary to achieve the desired melting characteristics. Also, fractionation and interesterification may be used to obtain fats of the desire melting range. Especially preferred are fats having relatively large proportions of polyunsaturated fatty acid moieties, such as canola and soybean oils. The fat compositions mentioned in Netherlands patent documents No. NL 143115, NL 178559, NL 155436, NL 149687, NL 155177, and European patent documents EP 41303, EP 209176, EP 249282, and EP 470658, the disclosures of which are incorporated by reference, are highly suitable. If a fat blend is used it is most preferred that it comprises at least 30%, more preferably at least 45% of polyunsaturated fatty acid moieties, based on the total weight amount of the fat in the fat based food product to promote cholesterol lowering.

The fat can be a single fat or a blend. The use of a fat composition comprising a considerable amount of PUFA (polyunsaturated fatty acid) rich triglycerides is in particular considered highly beneficial.

Non-digestible fats may also be sued as the fat source. Among the non-digestible fats are included polyol polyesters of C₈ to C₂₂ fatty acids such as sucrose polyester, sucrose polyethers, silicone oils/siloxanes, polycarboxylic acid esters, branched chain fatty acid triglycerides, neopentyl alcohol esters, dicarboxylic acid esters, jojoba oil and triglycerol ethers. Non-digestible fats may be used as from 0 to 100% of the fat, especially from 10 to 90%, and most especially from 25 to 75%.

Non-lipid fat replacers may also be used, to provide body to the product. These include protein-based fat replacers such as those described in Singer et al., U.S. Pat. No. 4,961,953 and cellulosic bulking agents such as microcrystalline cellulose and carboxymethyl cellulose.

Coloring agents, such as beta carotene, paprika, turmeric, annatto and yellow #5 and 6 and combinations thereof may be employed. The yellow color may desirably be used in combination with an opacifier like TiO₂. It has been found that providing an appropriate color may be important since phytoestrogen sources such as soy flour impart a brownish color.

Soy protein may be present in the compositions of the invention, particularly where it is present with the phytoestrogens which are added to the spread, as in soy flour. Other proteins can conveniently be in the form of milk protein from whole, skim or other low fat milk and may comprise whey proteins (with or without lactose), acid casein and caseinates. In addition or instead of soy, other vegetable protein, such as peanut protein, cottonseed protein and the like may also be used.

In addition to sodium chloride, flavor enhancers which may be employed include lactones, lipolyzed butter oils and started distillates, diacetyl, 2-octanone, butyric acid, hexanoic acid, and other fatty acids, esters of butyric acid, hexanoic acid, and other fatty acids, esters of butyric acid, delta-hydroxy acids and their glycerol esters and mixtures thereof.

Preservatives, such as benzoic acid, sorbic acid, phosphoric acid, lactic acid, acetic acid, hydrochloric acid and the soluble salts thereof may be used. Likewise, the emulsion may include antimicrobials such as potassium sorbate, sodium sorbate, potassium benzoate, sodium benzoate and phosphoric acid.

Antioxidants may include normal propyl gallagte, the tocopherols, including Vitamin E, butylated hydroxyanisole (BHA), butylated hydroxytoluene (BHT), nordihydorguaiaretic acid (NDGA), tertiary-butylhydroquinon (TBQH) and citric acid.

It will be appreciated that normally more hydrophobic additives will be added to the fat phase whereas more hydrophillic additives will normally be added to the aqueous phase.

The aqueous phase comprises water and, optionally other ingredients. A preferred ingredient is one or more gelling agents such as gelatin. Where the spread is a low fat spread, it is advantageous that the aqueous composition is gelled, which in some respects compensates for the lower amounts of fat in the product. It may be advantageous for the aqueous composition to be pre-gelled, i.e., gelled prior to combining the aqueous composition with the fat-continuos emulsion. Other suitable gelling agents include waxy maize starch such as Ultra-Tex 2, available from the National Starch and Chemical co., Bridgewater, N.J. or a rice starch such as Remyrise AC. A particularly effective combination of gelling agents has proved to be gelatin and waxy maize or rice starch. Other gelling agents include carrageenan, and a gelling hydrolyzed starch derivatives such as gelling maltodextrin, for example, Paselli maltodextrin SA2®.

The amount of gelling agent may lie between 0 and 30%, mostly between 0.1 and 25% based on the weight of the aqueous phase of the spread. If hydrolyzed starches are present, their level may be from 2-20%; other gelling agents may be used at levels of up to 10%, mostly 1-7%, most preferred 1.5-5%, all of these percentages being based on the weight of the aqueous phase.

Hydrocolloids which are thickening rather than gelling agents may also be used. Hydrocolloids are described in Zeitschrift fur Lebenmittletechnologie und Verfahrenstechnk 32 (1981) 6, pp. 253-256. Hydrocolloids in addition to those mentioned above include polysaccharides such as native and modified starches, cellulose derivatives, pectins, galleon, xanthan gum, agar, Danish agar, furcelleran, gum arabic, guar gum, locust bean gum, algin, and alginates.

Hydrocolloids will generally be used at levels of from 0.1 to 6%, based on total products. It will be appreciated that the gelling and thickening agents may be used in various combinations.

Additional ingredients which may be present in the aqueous phase include salt (particularly sodium chloride), preservatives, such as potassium sorbate, lactic and other acid, proteins, coloring agents, flavors, antimicrobials, antioxidants and vitamins, particularly water-soluble vitamins such as the B vitamins.

Addition of strong flavoring such as oregano and/or garlic, as well as spices can be important in masking off flour of phytoestrogen sources such as soy.

Proteins, water-soluble coloring agents, flavors, preservatives and antimicrobials and antioxidants useful in the aqueous composition are the same as those discussed above in connection with the fat phase, it being appreciated that generally the more hydrophillic additives are best placed in the aqueous phase.

Although melatonin may be added, compositions in which melatonin is essentially not present, is especially compositions in which melatonin are completely absent, are preferred.

Sources of phytoestrogens include Indian liquorice (Abrus precatorius); various species of Acacia spp. including, A. aneura, A. cibaria, A. longifolia, and A. oswaldii; ground nut (Apio tuberosa), ground bean (Arachis hypogea); milk vetch (Aastragalus edulis); marama bean (Bauhinia esculenta); sword bean (Cajanus cajan indicus); jack bean (Canavalia ensiformis); sword bean (Canavalia gladiata); seaside sword bean (Canavalia rosea); various Cassia spp. Including C. floribunda, C. laevigata, and C. occidentalis; carobbean (Ceratonia siliqua); chick pea (Cicer arietinum); yebnut (Cordeauxia edulis), various Crotalaria spp. Including C. laburnifolia, and C. pallida; cluster bean (Cyamopsis psoralioides); tallow tree (Detariaum senegalense); sword bean (Entada scandens); balu (Erythrina edulis); soyabean (Clycine max), inga (Ingaedulis), Polynesian chestnut (Inocarpus fagifer); hyacinth bean (Lablab purpureus); grass pea or Indian vetch (Lathyrus sativus); cyprus vetch (Lathyrus ochrus); lentil (Lens culinaris); jumping bean (Leucaneal eucocephala); various Lupinus spp. including L. albus, L. luteus, L. angustifolium, L. mutabilis, and L. cosentinii; ground bean (Macotylma geocarpa); hose gram (Macrotyloma uniflorum); alfalfa (Medicago sativa); velvet bean (Mucuna pruriens); yam beans (Pachyrhyzuz erosus, P. tuberosus); African locust bean (Parkia clappertoniana); Parkia speciosa; poil bean tree (Pentaclethra macrophylla); various Phaseolus spp. including P. acutifolium, P. vulgaris, P. luntus, P. coccineus, P. adenathus, P. angulris, P. aureus. P. calcaratus, P. mungo, and P. polystachyus; garden pean (Pisum sativumP); djenko bean (Pithecolobium lobatun); mesquite (various Prosopis spp.); goa bean (Psophocarpus scandens, P. tetragonolobus); various Psoralea spp.; Sesbania bispinosa; yam bean (Sphenostylis stenocarpa); tamarind (Tamarindus indicaP); fenugreek (Trigonella foenumgracum); vetches (various Vvia spp. including V. sativa, VC. Atropurpurea, V. ervilia, and V. monantha); broad bean (Vivia faba); black gram (Vigna mungo); various Vigna spp including V. radiata, V. aconitifolia, V. adanatha, V. angularus, V. tribolata, V. unbelata, and V. unguiculata; and earth pea (Voandzeia subterranea).

The balance of the spread is largely water, which may be incorporated at levels of up to 99.9% by weight, more generally form 10 to 98%, preferably from 20 to 97% by weight. Spreads according to the invention may be fat- or water-continuous.

| Ingredients | % |
|---|---|
| Oil phase | 40.00 |
| Canola Oil | 28.08 |
| Bean Oil | 2.77 |
| Partially Hydrogenated Bean Oil, | 8.70 |
| melting paint 42 C. | |
| Lecithin | 0.22 |
| Saturated Distilled Monoglyceride | 0.22 |
| (Iodine Value <5) | |
| Flavor | trace |
| Vitamin A | 0.01 |
| Aqueous phase | 60.00 |
| Water | 51.13 |
| Salt | 1.50 |
| Lactic Acid | 0.09 |
| Potassium Sorbate | 0.11 |
| Calcium Disodium EDTA | 0.01 |
| Pork Gelatin | 2.00 |
| Soylife micro (Soy germ flour) | 2.86 |
| Beta Tricalcium Phosphate | 1.88 |
| Xanthan gum | 0.10 |
| Artificial Color Yellow 5 | 0.04 |
| Titatnium Dioxide | 0.28 |
| Vitamin mix B6, B12, & E | 0.07 |
| total | 100.00 |

The spread is prepared by the following procedure:

The oil phase was prepared by heating the liquid oil and partially hydrogenated bean oil in a tank to 65° C. The emulsifiers, lecithin and monoglycerides were mixed and the mixture was held for 30 minutes to completely melt the fat crystals.

Vitamin A, flavor were added to the heated oil phase.

The aqueous phase was prepared by adding xanthan gum to the water at 40° C. in a tank. After hydrating the gum for 15 minutes, tricalcium phosphate was dispersed. All the other dry ingredients were added and mixed with a high shear mixer to obtain a homogeneous aqueous phase. The contents in the tank were batch pasteurized by heating to 80° C. and holding for 5 minutes and cooled to 55° C.

The fat and aqueous phases are mixed together at approximately 55° C. in a heated tank in a ratio of approximately 40 parts fat phase to 60 parts aqueous phase. This emulsion is water continuous. The emulsion is then passed through a cooled, scraped-surface heat exchanger (A-unit) where the emulsion is cooled to a temperature where the fat will begin to crystallize (few degrees C. below the alpha point 4° C. and the aqueous phase will begin to gel, if the aqueous phase has the gelling agents, and/or there is increase in viscosity if only thickening agents are present in the aqueous phase. The cooled emulsion is then passed into a slowly agitated, variable speed crystallizer (C*-unit) where the product is inverted from a water-continuous emulsion to a fat-continuous emulsion by quickly increasing the shaft speed. The inversion was aided by injecting 100% fat into the system. The C*-unit is referred to as the inverter unit. The invertor speed was 1000 rpm. The shaft speed in the invertor unit depends on its dimensions but normally varies from 200-2000 RPM. The fat continuous emulsion is passed into an additional C unit running at shaft speed of 300 RPM to provide gentle mixing while the fat continues to crystallize from the alpha to beta prime form.

Extra cooling capacity can be added to the process by including additional A-units. Extra residence time can be added to the process by including additional C-units.

Unless stated otherwise or required by context, the terms “fat” and “oil” are used interchangeably herein. Where a phase is said to constitute essentially the entire product, it is meant that such phase constitutes at least 98 wt. %, especially more than 99 wt. % of such product. Unless otherwise stated or required by context, percentages are by weight.

What is claimed is:

1. A edible, plastic water-in-oil spread comprising: added phytoestrogens.

2. The spread according to claim 1 wherein said phytoestrogens are isoflavones.

3. The spread according to claim 2 wherein said phytoestrogens are selected from the group consisting of genistein, genistin, daidzein, glycitein, formonenetin, biochanin A and saponins.

4. The spread according to claim 1 wherein said phytoestrogens are obtained from soy.

5. The spread according to claim 4 wherein said phytoestrogens are added in the form of soy flour.

6. The spread according to claim 1 further comprising added calcium salt.

7. An edible, plastic spread comprising phytoestrogens at a level of at least 0.01 wt. % and at least 0.25 wt. % calcium.

8. An edible, plastic spread comprising phytoestrogens at a level of at least 0.01 wt. % and at least two of the following vitamins: D, E, B6 and B12.

9. An edible, plastic spread comprising one or more isoflavones at a level of at least 0.01 wt. %, at least 0.4% soy protein, at least 0.25% calcium salt,

10. An edible, plastic spread comprising one or more isoflavones at a level of at least 0.05 wt. %.

11. An edible, plastic spread comprising one or more isoflavones at a level of at least 0.05 wt. % and less than 2 wt. % protein.

12. The spread according to claim 11 wherein said protein is present at a level of less than 1.5 wt. %.

13. The spread according to claim 12 further comprising saponins at a level of at least about 0.05% and tocopherols at a level of at least 0.001 wt. %.

14. The spread according to claim 13 further comprising at least 5 wt. % polyunsaturated fatty acid moieties.

15. The spread according to claim 14 wherein said polyunsaturated fatty acid moieties are present at a level of at least 10 wt. %.

16. The spread according to claim 15 wherein said polyunsaturated fatty acid moieties are present at a level of up to 20 wt %.

17. The spread according to claim 1 wherein the phytoestrogen comprises genistein.

18. The spread according to claim 7 wherein the phytoestrogen comprises genistein.

19. The spread according to claim 9 wherein the phytoestrogen comprises genistein.

20. The spread according to claim 1 further comprises an artificial yellow colorant.

21. The spread according to claim 8 wherein two or more of the following vitamins are present. Vitamin E, at least 0.01 wt. %; vitamin B₆ at least 0.001 wt. %; Vitamin B₁₂ at least 0.001 wt. %.

22. The spread according to claim 8 wherein two or more of the following vitamins are present. Vitamin E, at least 0.05%; vitamin B at least 0.002%; Vitamin B₁₂ at least 0.002%.
